(19) United States
(12) Patent Application Publication
(10) Pub. No.: US 2025/0213206 A1
(43) Pub. Date: Jul. 3, 2025

(54) MEDICAL IMAGE PROCESSING APPARATUS, X-RAY DIAGNOSTIC APPARATUS, AND STORAGE MEDIUM STORING MEDICAL IMAGE PROCESSING PROGRAM

(71) Applicant: CANON MEDICAL SYSTEMS CORPORATION, Otawara-shi (JP)

(72) Inventors: Shingo ABE, Nasushiobara (JP); Yuki KOIKE, Otawara (JP)

(73) Assignee: CANON MEDICAL SYSTEMS CORPORATION, Otawara-shi (JP)

(21) Appl. No.: 19/000,115

(22) Filed: Dec. 23, 2024

(30) Foreign Application Priority Data
Dec. 28, 2023 (JP) 2023-222421

(51) Int. Cl. A61B 6/12; A61B 6/46; G06T 3/40; G06T 7/33; G06T 7/73

(52) U.S. Cl. CPC A61B 6/12; A61B 6/463; G06T 3/40; G06T 7/33; G06T 7/73; G06T 2207/10016; G06T 2207/10116; G06T 2207/20101

(57) ABSTRACT

A medical image processing apparatus according to an embodiment includes processing circuitry. The processing circuitry sequentially acquires an X-ray image including a device inserted into a subject. Then, the processing circuitry detects a distal end of the device on the X-ray image. Then, the processing circuitry determines a position on the device distant from the distal end, as a base point. Then, the processing circuitry generates an image in which at least a position of the base point is located at the same position or within a predetermined range including the position, among a plurality of the X-ray images, and displays the image on the display.

### CROSS-REFERENCE TO RELATED APPLICATIONS

This application is based upon and claims the benefit of priority from Japanese Patent Application No. 2023-222421, filed Dec. 28, 2023, the entire contents of which are incorporated herein by reference.

### FIELD

Embodiments described herein relate generally to a medical image processing apparatus, an X-ray diagnostic apparatus, and a storage medium storing a medical image processing program.

### BACKGROUND

In coronary artery interventional radiology (IVR), there is a technique for detecting catheter marker pairs in an X-ray fluoroscopic image or an X-ray computed tomography (CT) image, aligning each image so as to cancel catheter movement due to heartbeat and breathing, and displaying a video. For example, a heart is constantly pumping, and thus, a catheter inserted into the heart moves according to the pumping. An operator wishes to observe a movement of the catheter caused by his/her own operation, and therefore, it is useful if it is possible to cancel the movement of the catheter caused by the heartbeat and the like. However, such a technique requires a marker pair and cannot be applied when a device such as a guidewire, a catheter, and a coil does not have the marker pair.

There is a technique for improving visibility of X-ray image data including a device regardless of the presence or absence of the marker pair. In such a technique, two characteristic points in the X-ray image data are extracted as substitutes for the marker pair, and positions of a plurality of pieces of X-ray image data are corrected using the two characteristic points as reference points. In this case, however, the position and the angle of a distal region of a guidewire are substantially fixed among the plurality of pieces of X-ray image data. Therefore, in operating a device, such as a guidewire, where a movement of a distal end is important, a display method in which the distal end is fixed in this manner is not considered appropriate.

### BRIEF DESCRIPTION OF THE DRAWINGS

FIG. 1 is a block diagram illustrating an example of a configuration of a medical information processing system according to a first exemplary embodiment;

FIG. 2 is a block diagram illustrating an example of a configuration of an X-ray diagnostic apparatus according to the first exemplary embodiment;

FIG. 3 is a flowchart illustrating an example of processing in the medical information processing system according to the first exemplary embodiment;

FIG. 4A to FIG. 4F are diagrams illustrating an example of an X-ray image according to the first exemplary embodiment;

FIG. 5 is a flowchart illustrating device line detection processing according to the first exemplary embodiment;

FIG. 6 is a flowchart illustrating device base point determination processing according to the first exemplary embodiment;

FIG. 7 is a diagram for describing display region setting processing according to the first exemplary embodiment;

FIG. 8 is a flowchart illustrating an example of processing in a medical information processing system according to a second exemplary embodiment;

FIG. 9 is a diagram illustrating an example of a display region according to a third exemplary embodiment; and

FIG. 10 is a diagram illustrating an example of X-ray image data according to the third exemplary embodiment.

### DETAILED DESCRIPTION

A medical image processing apparatus according to an exemplary embodiment includes an acquisition unit, a detection unit, a base point determination unit, and a display control unit. The acquisition unit sequentially acquires an X-ray image including a device inserted into a subject. The detection unit detects a distal end of the device on the X-ray image. The base point determination unit determines a position on the device away from the distal end, as a base point. The display control unit generates an image in which at least a position of the base point is located at the same position or within a predetermined range including the position, among a plurality of X-ray images, and displays the image on a display unit.

Various Embodiments will be described hereinafter with reference to the accompanying drawings.

### First Exemplary Embodiment

First, a first exemplary embodiment will be described. In the first exemplary embodiment, a medical information processing system including a medical image processing apparatus will be described as an example.

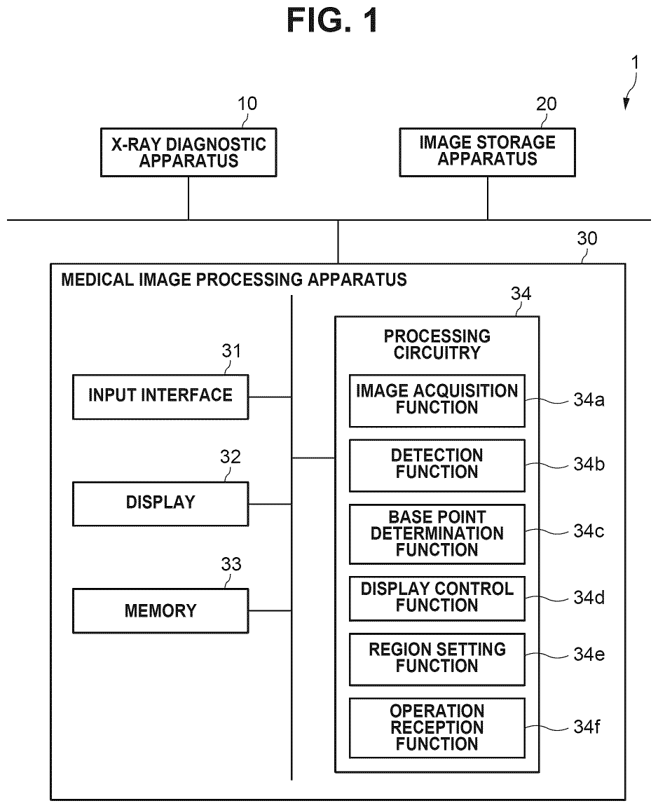

FIG. 1 is a block diagram illustrating an example of a configuration of a medical information processing system 1 according to the first exemplary embodiment. As illustrated in FIG. 1, the medical information processing system 1 according to the first exemplary embodiment includes an X-ray diagnostic apparatus 10, an image storage apparatus 20, and a medical image processing apparatus 30. The X-ray diagnostic apparatus 10, the image storage apparatus 20, and the medical image processing apparatus 30 are connected to each other via a network.

The X-ray diagnostic apparatus 10 collects X-ray image data from a subject P. For example, the X-ray diagnostic apparatus 10 collects a plurality of pieces of X-ray image data from the subject P, and transmits the plurality of pieces of collected X-ray image data to the image storage apparatus 20 or the medical image processing apparatus 30. A configuration of the X-ray diagnostic apparatus 10 will be described below.

The image storage apparatus 20 stores the plurality of pieces of X-ray image data collected by the X-ray diagnostic apparatus 10. For example, the image storage apparatus 20 is realized by a computer device such as a server device. In the first exemplary embodiment, the image storage apparatus 20 acquires the plurality of pieces of X-ray image data from the X-ray diagnostic apparatus 10 via the network, and stores the plurality of pieces of acquired X-ray image data in a memory provided within or outside the image storage apparatus 20.

The medical image processing apparatus 30 acquires a plurality of pieces of time-series X-ray image data via the network, and executes various kinds of processing by using the plurality of pieces of acquired X-ray image data. For example, the medical image processing apparatus 30 is realized by a computer device such as a workstation. In the first exemplary embodiment, the medical image processing apparatus 30 acquires the plurality of pieces of X-ray image data from the X-ray diagnostic apparatus 10 or the image storage apparatus 20 via the network.

As illustrated in FIG. 1, the medical image processing apparatus 30 includes an input interface 31, a display 32, a memory 33, and processing circuitry 34.

The input interface 31 is realized by a trackball, a switch, a button, a mouse, and a keyboard for issuing various types of instructions and performing various types of settings, a touchpad that allows an input operation by touching an operation surface, a touch screen in which a display screen and a touch pad are integrated, contact input circuitry using an optical sensor, voice input circuitry, and the like. The input interface 31 converts an input operation received from an operator into an electrical signal and outputs the signal to the processing circuitry 34. The input interface 31 is not limited to an interface equipped with a physical operation part such as a mouse and a keyboard. For example, an example of the input interface 31 may be electrical signal processing circuitry configured to receive an electrical signal corresponding to an input operation from an external input device provided separately from the medical image processing apparatus 30 and to output the electrical signal to the processing circuitry 34.

The display 32 displays various types of information. For example, the display 32 displays graphical user interface (GUI) for receiving an instruction from the operator, and various types of X-ray image data. For example, the display 32 is a liquid crystal display or a cathode ray tube (CRT) display. The display 32 is an example of a display unit.

The memory 33 is realized by, for example, a semiconductor memory element such as a random access memory (RAM) or a flash memory, a hard disk, and an optical disk. For example, the memory 33 stores the plurality of pieces of X-ray image data acquired from the image storage apparatus 20. For example, the memory 33 stores a program for each circuit included in the medical image processing apparatus 30 to implement a function. The memory 33 is an example of a storage unit.

The processing circuitry 34 implements a function of controlling the medical image processing apparatus 30 in an integrated manner. A processor of the processing circuitry 34 reads and executes a medical image processing program stored in the memory 33 to implement an image acquisition function 34a, a detection function 34b, a base point determination function 34c, a display control function 34d, a region setting function 34e, and an operation reception function 34f. Each of these functions 34a to 34f is stored in the memory 33 in the form of a program.

The image acquisition function 34a includes a function of sequentially acquiring the X-ray image including a device inserted into the subject P. The detection function 34b includes a function of detecting a distal end of the device on the X-ray image. The base point determination function 34c includes a function of determining a position on the device away from the distal end of the device, as a base point. The display control function 34d includes a function of generating an image in which at least a position of the base point of the device is located at the same position or within a predetermined range including the position, among a plurality of X-ray images, and displaying the image on the display 32. The region setting function 34e includes a function of setting a display region for displaying at least the distal end to the base point of the device. The operation reception function 34f includes a function of receiving an operation by an operator to change the position of the base point. The practitioner is an example of a user. The display control function 34d includes a function of generating an image of a display region R1 and displaying such an image on the display 32 so that, for each X-ray image acquired sequentially, the base point of the device is located at the same position or within a predetermined range including the position within the display region R1.

In the medical image processing apparatus 30 illustrated in FIG. 1, each processing function is stored in the memory 33 in the form of a computer-executable program. The processing circuitry 34 is a processor configured to implement a function corresponding to each program by reading the program from the memory 33 and executing the program. In other words, the processing circuitry 34 that has read each program has a function corresponding to the read program. While it is described that the image acquisition function 34a, the detection function 34b, the base point determination function 34c, the display control function 34d, the region setting function 34e, and the operation reception function 34f are implemented by the single processing circuitry 34 in FIG. 1, the processing circuitry 34 may be configured by combining a plurality of independent processors, and each processor may execute a program to implement each function.

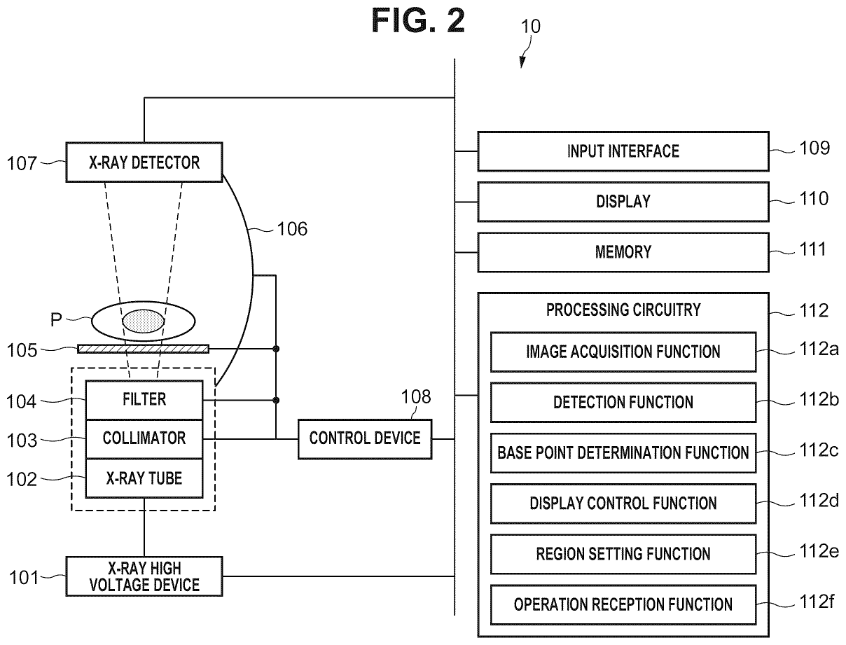

FIG. 2 is a block diagram illustrating an example of a configuration of the X-ray diagnostic apparatus 10 according to the first exemplary embodiment. As illustrated in FIG. 2, the X-ray diagnostic apparatus 10 includes an X-ray high voltage device 101, an X-ray tube 102, a collimator 103, a filter 104, a top board 105, a C-arm 106, an X-ray detector 107, a control device 108, an input interface 109, a display 110, a memory 111, and a processing circuitry 112.

The X-ray high voltage device 101 supplies a high voltage to the X-ray tube 102 under the control of the processing circuitry 112. For example, the X-ray high voltage device 101 includes electrical circuitry including a transformer and a rectifier, and includes a high voltage generation device configured to generate a high voltage to be applied to the X-ray tube 102, and an X-ray control device configured to control an output voltage corresponding to the X-rays emitted from the X-ray tube 102. The high voltage generation device may be of a transformer type or an inverter type.

The X-ray tube 102 is a vacuum tube including a cathode (filament) configured to generate a thermal electron and an anode (target) configured to generate X-rays when struck by the thermal electron. The X-ray tube 102 generates X-rays by using the high voltage supplied from the X-ray high voltage device 101 and emitting the thermal electron toward the anode from the cathode.

The collimator (also referred to as an X-ray aperture device) 103 includes, for example, four slidable diaphragm blades. The collimator 103 narrows the X-rays generated by the X-ray tube 102 by sliding the diaphragm blades, and irradiates the subject P with the X-rays. Here, the diaphragm blades are plate-like members made of lead or the like, and are disposed near an X-ray irradiation port of the X-ray tube 102 to adjust an irradiation range of the X-rays.

To reduce an exposure dose to the subject P and improve image quality of the X-ray image data, the filter 104 changes the quality of the X-rays passing through the filter 104 depending on its material and thickness to reduce a soft ray component easily absorbed by the subject P and reduce a high energy component causing a decrease in contrast of the X-ray image data. The filter 104 changes the X-ray dose and the irradiation range depending on its material, thickness, position, and the like, and attenuates the X-rays so that the X-rays emitted from the X-ray tube 102 toward the subject P have a predetermined distribution.

The top board 105 is a plate on which the subject P lies, and is placed on a couch (not illustrated). It should be noted that the subject P is not included in the X-ray diagnostic apparatus 10.

The C-arm 106 holds the X-ray tube 102, the collimator 103, the filter 104, and the X-ray detector 107 so as to face each other with the subject P being placed therebetween. It should be noted that in FIG. 2, the X-ray diagnostic apparatus 10 is described as being a single-plane apparatus, but the exemplary embodiment is not limited thereto, and may be a bi-plane apparatus.

The X-ray detector 107 is, for example, an X-ray flat panel detector (FPD) having detection elements arranged in a matrix. The X-ray detector 107 detects the X-rays emitted from the X-ray tube 102 and having transmitted through the subject P, and outputs a detection signal corresponding to the amount of detected X-rays to the processing circuitry 112. The X-ray detector 107 may be an indirect conversion type detector having a grid, a scintillator array, and a photosensor array, or may be a direct conversion type detector having a semiconductor element that converts incident X-rays into an electrical signal.

The control device 108 includes a drive mechanism such as a motor and an actuator, and a circuit configured to control the mechanism. The control device 108 controls the operations of the collimator 103, the filter 104, the top board 105, the C-arm 106, and the like under the control of the processing circuitry 112. For example, the control device 108 controls the irradiation range of the X-rays emitted onto the subject P by adjusting an opening rate of the diaphragm blades of the collimator 103. The control device 108 controls the distribution of the dose of X-rays emitted onto the subject P by adjusting the position of the filter 104. For example, the control device 108 rotates and moves the C-arm 106 and moves the top board 105.

The input interface 109 is realized by a trackball, a switch, a button, a mouse, and a keyboard for issuing various types of instructions and performing various types of settings, a touchpad that allows an input operation by touching an operation surface, a touch screen in which a display screen and a touch pad are integrated, non-contact input circuitry using an optical sensor, voice input circuitry, and the like. The input interface 109 converts an input operation received from an operator into an electrical signal and outputs the signal to the processing circuitry 112. The input interface 109 is not limited to an interface equipped with a physical operation part such as a mouse and a keyboard. For example, an example of the input interface 109 may be electrical signal processing circuitry configured to receive an electrical signal corresponding to an input operation from an external input device provided separately from the X-ray diagnostic apparatus 10 and to output the electrical signal to the processing circuitry 112.

The display 110 displays various types of information. For example, the display 110 displays a GUI for receiving an instruction from the operator, and various types of X-ray image data. For example, the display 110 is a liquid crystal display or a CRT display.

The memory 111 is realized by, for example, a semiconductor memory element such as a RAM or a flash memory, a hard disk, and an optical disk. The memory 111 receives and stores, for example, the X-ray image data collected by the processing circuitry 112. The memory 111 also stores a program corresponding to various types of functions read and executed by the processing circuitry 112.

The processing circuitry 112 implements the function of controlling the X-ray diagnostic apparatus 10 in an integrated manner. Some or all of the functions 34a to 34f of the processing circuitry 34 of the medical image processing apparatus 30 may be implemented by the processing circuitry 112 of the X-ray diagnostic apparatus 10. In the first exemplary embodiment, as illustrated in FIG. 1 and FIG. 2, an example is described in which all of the functions 34a to 34f of the processing circuitry 34 of the medical image processing apparatus 30 are implemented also by the processing circuitry 112 of the X-ray diagnostic apparatus 10. In such a case, a processor of the processing circuitry 112 reads and executes a medical image processing program stored in the memory 111 to implement an image acquisition function 112a, a detection function 112b, a base point determination function 112c, a display control function 112d, a region setting function 112e, and an operation reception function 112f. Each of these functions 112a to 112f is stored in the memory 33 in the form of a program.

The image acquisition function 112a includes a function of sequentially acquiring an X-ray image that is based on a detection signal output by the X-ray detector 107 and that includes the device inserted into the subject P. The detection function 112b includes a function of detecting the distal end of the device on the X-ray image. The base point determination function 112c includes a function of determining a position on the device away from the distal end of the device, as a base point. The display control function 112d includes a function of generating an image in which at least the position of the base point of the device is located at the same position or within a predetermined range including the position, among the plurality of X-ray images, and displaying the image on the display 110. The region setting function 112e includes a function of setting a display region for displaying at least the distal end to the base point of the device. The operation reception function 112f includes a function of receiving an operation by the practitioner to change the position of the base point.

In the X-ray diagnostic apparatus 10 illustrated in FIG. 2, each processing function is stored in the memory 111 in the form of a computer-executable program. The processing circuitry 112 is a processor configured to implement a function corresponding to each program by reading the program from the memory 111 and executing the program. In other words, the processing circuitry 112 that has read each program has a function corresponding to the read program. While it is described that the image acquisition function 112a, the detection function 112b, the base point determination function 112c, the display control function 112d, the region setting function 112e, and the operation reception function 112f are implemented by the single processing circuitry 112 in FIG. 2, the processing circuitry 112 may be configured by combining a plurality of independent processors, and each processor may execute a program to implement each function.

The term “processor” used in the above description refers to circuitry such as a central processing unit (CPU), graphics processing unit (GPU), and application specific integrated circuit (ASIC), as well as a programmable logic medical device (for example, a simple programmable logic device (SPLD)), complex programmable logic device (CPLD), and field programmable gate arrays (FPGA). The processor implements a function by reading and executing a program stored in the memory 33 or the memory 111. Instead of storing the program in the memory 33 or the memory 111, a configuration may be such that the program may be directly incorporated into circuitry of the processor. In such a case, the processor implements the function by reading and executing the program incorporated in the circuitry. The processor according to the first exemplary embodiment is not limited to a processor configured as a single circuit, but may be a processor configured by combining a plurality of independent circuits to implement its functions.

Processing performed by the medical image processing apparatus 30 according to the first exemplary embodiment will be described in detail below.

First, the image acquisition function 112a in the X-ray diagnostic apparatus 10 acquires a plurality of pieces of X-ray image data. For example, the image acquisition function 112a continuously or intermittently irradiates the heart of the subject P with a device inserted into the coronary artery with X-rays for a period according to an instruction from the operator. At this time, the X-ray detector 107 detects the X-rays passing through the heart of the subject P, and outputs a detection signal corresponding to the detected X-ray amount to the processing circuitry 112.

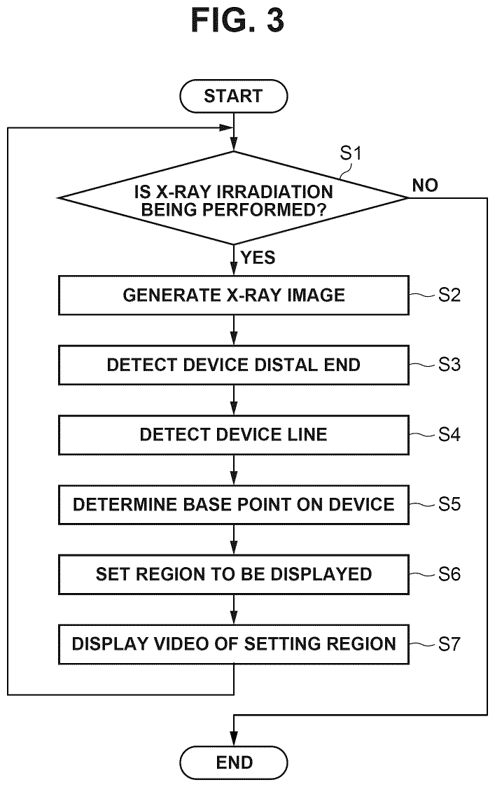

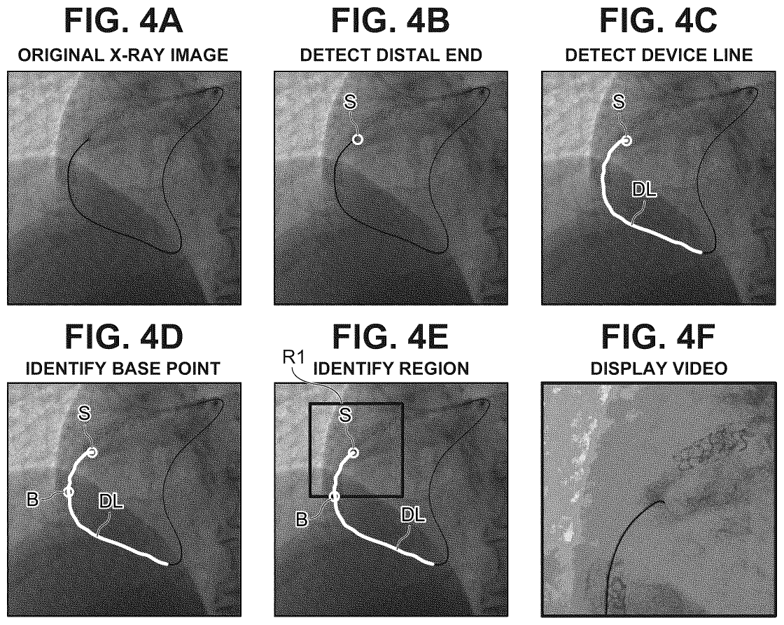

FIG. 3 is a flowchart illustrating an example of processing in the medical information processing system 1 according to the first exemplary embodiment. FIG. 4A to FIG. 4F are diagrams illustrating an example of the X-ray image according to the first exemplary embodiment. With reference to FIG. 3 and FIGS. 4A to 4F, an outline of the processing performed by the medical information processing system 1 will be described.

In step S1, the X-ray diagnostic apparatus 10 determines whether X-ray irradiation is being performed. For example, the operation reception function 112f of the X-ray diagnostic apparatus 10 may determine whether an operation for issuing an instruction for X-ray imaging of the subject P is performed on the input interface 109. Alternatively, the control device 108 of the X-ray diagnostic apparatus 10 may determine whether the X-ray tube 102 is emitting the X-rays.

If the X-ray irradiation is being performed (YES in step S1), the processing proceeds to step S2. If the X-ray irradiation is not being performed (NO in step S1), the X-ray diagnostic apparatus 10 ends the processing.

In step S2, the image acquisition function 112a of the X-ray diagnostic apparatus 10 generates X-ray image data based on the detection signal received from the X-ray detector 107, and stores the generated X-ray image data in the memory 111. The processing circuitry 112 transmits the generated X-ray image data to the image storage apparatus 20 or the medical image processing apparatus 30. FIG. 4A illustrates an original X-ray image.

In step S3, the image acquisition function 34a of the medical image processing apparatus 30 sequentially acquires the X-ray image data from the X-ray diagnostic apparatus 10 or the image storage apparatus 20 and stores the X-ray image data in the memory 33. The detection function 34b detects a distal end S of the device from the X-ray image data. Here, for example, existing distal end detection processing is performed (see the distal end S in FIG. 4B).

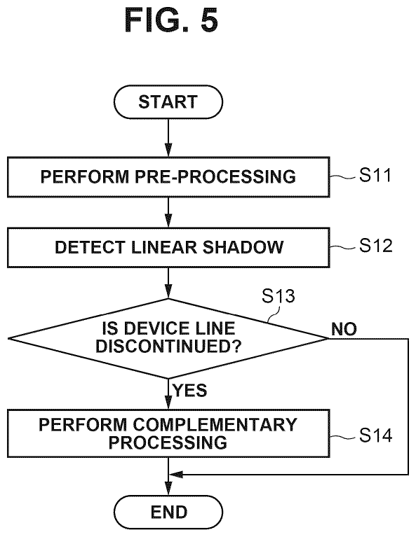

In step S4, the detection function 34b of the medical image processing apparatus 30 detects a device line DL, which is a linear shape of the device, from the X-ray image data. Here, for example, existing linear shadow detection processing is performed (see the device line DL in FIG. 4C). Device line detection processing will be described in detail below with reference to FIG. 5.

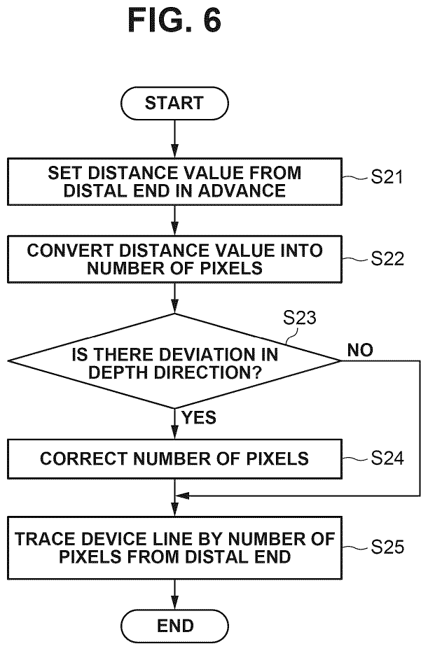

In step S5, the base point determination function 34c of the medical image processing apparatus 30 determines a base point B of the device from the X-ray image data. The base point determination function 34c determines, as the base point B, a position in the X-ray image data at a predetermined distance along the device line DL from the distal end S of the detected device toward a terminal end at the practitioner's hand (see the base point B in FIG. 4D). Base point determination processing will be described in detail below with reference to FIG. 6.

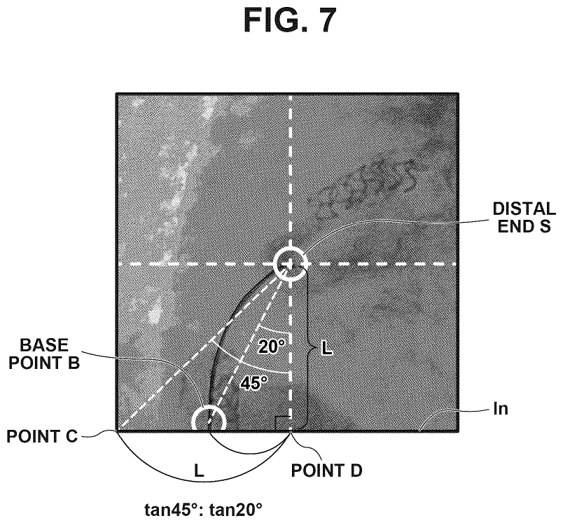

In step S6, the region setting function 34e of the medical image processing apparatus 30 sets the display region R1 that is a region to be displayed on the display 32, of the X-ray image data. The region setting function 34e sets the display region R1 based on a positional relationship between the distal end and the base point of the device the first time. From the second time onwards, the region setting function 34e sets the display region R1 based on the position of the base point B of the device (see the display region R1 in FIG. 4E). Display region setting processing will be described in detail below with reference to FIG. 7.

In step S7, the display control function 34d of the medical image processing apparatus 30 displays, on the display 32, an image of the set display region R1 of the X-ray image data. More specifically, the display control function 34d generates the image of the display region R1 for each X-ray image data acquired sequentially so that the base point B is located at the same position or within a predetermined range including the position within the display region R1, and displays the image on the display 32 (see FIG. 4F). As illustrated in FIG. 4F, the display control function 34d may enlarge and display the image of the set display region R1.

The display control function 34d may display the image of the display region R1, together with the X-ray image data, on the display 32. More specifically, the display control function 34d may display the image of the display region R1 and the X-ray image data side by side (for example, display the images of FIG. 4E and FIG. 4F side by side) on the display 32. The display control function 34d may superimpose the image of the display region R1 on a predetermined position (for example, a region corresponding to the display region R1 in the original X-ray image) of the original X-ray image (see FIG. 4A), and display the image on the display 32. Alternatively, the device line detection in step S4 may be performed, and then, the device distal end detection in step S3 may be performed. While the processing in steps S3 to S7 is described as being performed by the medical image processing apparatus 30, the processing may be performed by the X-ray diagnostic apparatus 10. In other words, the entire processing in steps S1 to S7 may be performed by either the X-ray diagnostic apparatus 10 or the medical image processing apparatus 30.

According to the above, in each X-ray image data, the display region R1 is set based on the position of the base point B of the device, and the image of the display region R1 is displayed so that the base point B is located at the same position or within a predetermined range including the position within the display region R1. Therefore, as a result of the position of the base point B of the device being fixed at approximately the same position, the practitioner who continuously observes the image of the display region R1 generated for each sequentially acquired X-ray image can check serial images of the device in which the distal end S of the device moves in accordance with an intent of the practitioner, while an unnecessary movement of the device caused by heartbeat and breathing is suppressed. This allows the practitioner to easily check a movement of the distal end S of the device.

FIG. 5 is a flowchart illustrating the device line detection processing according to the first exemplary embodiment. FIG. 5 is a flowchart for describing in detail the processing of step S4 in FIG. 3.

In step S11, the detection function 34b of the medical image processing apparatus 30 performs pre-processing on the X-ray image data to suppress erroneous detection. The pre-processing includes, for example, threshold processing, noise reduction processing, and frequency separation processing.

In step S12, the detection function 34b detects the device line DL from the X-ray image data by using, for example, linear shadow detection.

In step S13, the detection function 34b determines whether the device line DL is discontinued. If the device line DL is discontinued (YES in step S13), the processing proceeds to step S14. If the device line DL is not discontinued (NO in step S13), the detection function 34b ends the device line detection processing.

In step S14, the detection function 34b complements the discontinued device line DL. For example, when the device line DL is discontinued due to an influence of noise or a background, complementary processing such as morphological processing or spline processing is performed. Then, the detection function 34b ends the device line detection processing.

The detection function 34b may extract a linear shape close to the device line DL detected in a previous X-ray image by using pattern matching processing or the like. This can improve the accuracy of device line detection. When the width of a curve assumed to be the device has a plurality of pixels, the detection function 34b may identify the device line DL by a center line that is the center of the width of the curve, a shortest path included in the curve (for example, the contour of an inner circumference of the curve having a width), or the like. Furthermore, the detection function 34b may learn shapes of a plurality of device lines DL previously detected by deep learning, and select, as the device line DL, a curve most likely to be the device line from a plurality of curves extracted from a new X-ray image.

FIG. 6 is a flowchart illustrating the device base point determination processing according to the first exemplary embodiment. FIG. 5 is a flowchart for describing in detail the processing of step S5 in FIG. 3.

In step S21, the base point determination function 34c of the medical image processing apparatus 30 sets, in the memory 33, a distance value [mm] from the distal end S of the device in advance. The distance value (predetermined distance) from the distal end S is determined in advance according to at least one of an X-ray imaging condition and a protocol. For example, the device to be used is determined depending on a procedure to be performed next, and the distance value from the distal end S to the base point B is determined depending on the device. The distance value set in the memory 33 may be changed by the practitioner as appropriate. The distance value is an example of the predetermined distance.

In step S22, the base point determination function 34c reads the distance value determined in advance from the memory 33, and converts the distance value into the number of pixels on the X-ray image. A conversion rate from the distance value to the number of pixels is calculated according to a field of view (FOV, X-ray irradiation region) of the X-ray diagnostic apparatus 10, geometric magnification, digital zoom, and the like. The geometric magnification means that a magnification rate of the X-ray image changes depending on a positional relationship between the X-ray tube 102, the X-ray detector 107, and the subject P. The digital zoom is a complement to an optical zoom, and enlarges or reduces an image by using digital processing.

In step S23, the base point determination function 34c determines whether there is a deviation in a depth direction of the device line DL. The presence or absence of the deviation in the depth direction can be determined based on three-dimensional (3D) information such as biplane irradiation, two-way irradiation, and a 3D volume. If there is the deviation in the depth direction (YES in step S23), the processing proceeds to step S24. If there is no deviation in the depth direction (NO in step S23), the processing proceeds to step S25.

In step S24, the base point determination function 34c corrects the number of pixels according to the deviation in the depth direction of the device line DL. When a device line is deviated in the depth direction, the number of pixels on the X-ray image is smaller as a result of the image being projected onto an X-ray detection surface compared to when the device line is not deviated (is on a plane perpendicular to an imaging direction, i.e., on a plane parallel to the X-ray detection surface). Therefore, the base point determination function 34c may convert the predetermined distance into the number of pixels on the X-ray image based on the 3D information of the device.

In step S25, the base point determination function 34c determines the base point B based on the distal end S and the number of pixels. More specifically, the base point determination function 34c determines the base point B by tracing, by the number of pixels, the device line DL from the distal end S to the terminal end of the device.

The base point determination function 34c may determine a first base point (base point B) from the distal end S of the device along the device line DL, and determine a position at a distance different from the above-described distance value (position farther from the distal end S than the base point B) as a second base point. In such a case, the region setting function 34e sets a display region R1 for each of the sequentially acquired X-ray images based on the positions of the first base point and the second base point. Then, the display control function 34d positions the second base point at approximately the same position in the display region R1, while changing the position of the first base point in accordance with the amount of change of the distal end S, to generate the image of the display region R1. As a result, it is possible to stabilize display of a moving image.

FIG. 7 is a diagram for describing the display region setting processing according to the first exemplary embodiment. As illustrated in FIG. 7, the region setting function 34e sets the display region R1 such that, for example, the distal end S is located at the center of the region and the base point B is located at an end of the region. FIG. 7 illustrates an example in which the display region R1 is a square and the base point B is located on the bottom side of the square. When the display region R1 is a square, in particular, as illustrated in FIG. 7, the region setting function 34e first draws a horizontal straight line In passing through the base point B in the X-ray image. Next, a line perpendicular to the line In and a line intersecting the line In at 45° are drawn from the distal end S. When intersections of these two lines and the horizontal line In are points D and C, respectively, a square with each side being twice the length of a line segment CD is set as the display region R1. Thus, the first processing is described above. When the length of the line segment CD is L, in the example illustrated in FIG. 7, the base point B is a point away to the left from the point D being the midpoint of the bottom side of the square by L tan 20°.

From the second time onwards, the image of the display region R1 is generated so that the base point B is located at the same position or within a predetermined range including the position within the display region R1. If the base point B is fixed at the same position within the display region R1, the base point determination function 34c sets the display region R1 so that, when the next X-ray image is acquired, the base point B is at the same position as the base point B set the first time. At this time, the length of one side of the display region R1 is maintained at 2 L, and the length of a line segment BD is maintained at Ltan 20°. Therefore, even in the second display region setting processing, the base point B is located at the same position within display region R1 as the position illustrated in FIG. 7. On the other hand, the position of the distal end S changes within the display region R1 in response to the movement of the device.

The base point determination function 34c may change the distance value in accordance with the amount of change of the device in the display region R1. If a slope of the line connecting the distal end S and the base point B varies significantly between the X-ray images (frames), an end point of the variation of the base point B (position before or after the variation) or the center point of the variation may be used as the base point B. Further, if the slope of the line connecting the distal end S and the base point B varies significantly, the base point determination function 34c may suggest, to the practitioner via the display 32, that the base point B be changed to a position with less variation, and may accept the change in accordance with an input instruction from the practitioner.

It is desirable that the display control function 34d updates the images of the distal end S, the display region R1, and the like for each frame as needed, while maintaining the position of the base point B on the display region R1 fixed. The display control function 34d may automatically update the images of the distal end S, display region R1, and the like at predetermined time intervals or when the amount of change in the angle of the slope of the straight line connecting the distal end S and the base point B exceeds a threshold value. The variation in the angle of the slope occurs due to a change in the position of the device within a blood vessel. Further, the display control function 34d may update the images of the distal end S, the display region R1, and the like according to latest detection results in response to a device operation by the practitioner.

Further, in the above description, the example is described in which the base point B is located on the bottom side of the square that is the display region R1, but depending on the positional relationship between the distal end S and the base point B, the base point B may be located on the top side, right side, or left side of the square, or the base point B may be located at any of the four vertices of the square. In such a case as well, the same processing as above is performed.

According to the first exemplary embodiment, even in a device such as a guide wire not including the marker pair, it is possible to eliminate an unnecessary movement due to heartbeat and breathing. Therefore, it is easier to check the movement of the distal end S of the device, and it is possible to assist the practitioner in operating the device.

### Second Exemplary Embodiment

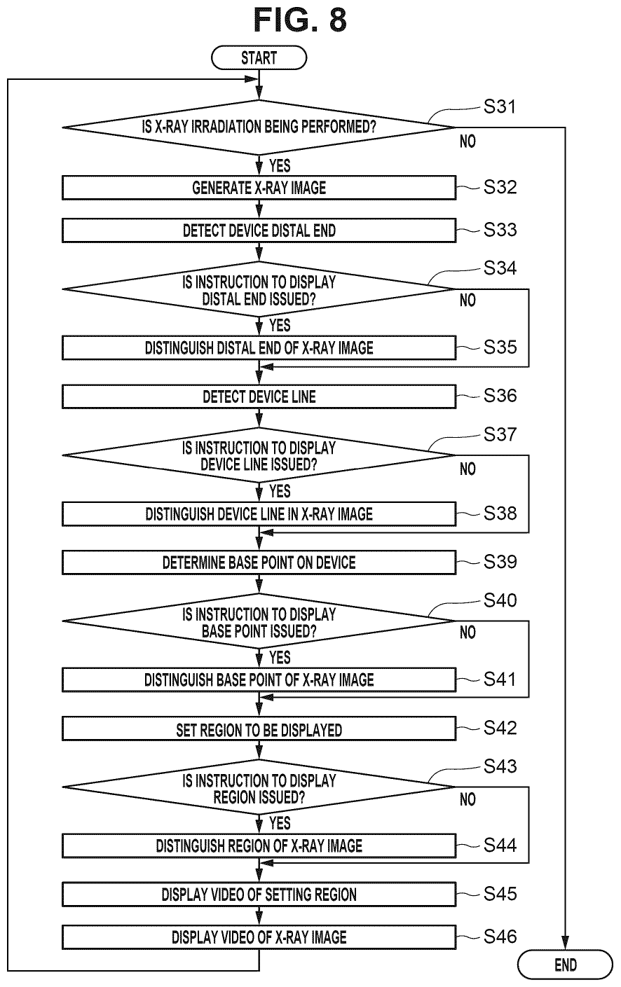

FIG. 8 is a flowchart illustrating an example of processing in a medical information processing system 1 according to a second exemplary embodiment. In the second exemplary embodiment, a region set from X-ray image data is displayed, and the entire X-ray image data including a distinguished portion is also displayed. In other words, when the X-ray image data is displayed, a portion of the data, namely, the distal end S of the device, the device line DL, the base point B, and the display region R1 are selectively displayed or hidden. Contents overlapping with the description of FIG. 3 will be omitted.

In step S31, the X-ray diagnostic apparatus 10 determines whether the X-ray irradiation is being performed. If the X-ray irradiation is being performed (YES in step S31), the processing proceeds to step S32. If the X-ray irradiation is not being performed (NO in step S31), the X-ray diagnostic apparatus 10 ends the processing.

In step S32, the image acquisition function 112a of the X-ray diagnostic apparatus 10 generates X-ray image data based on a detection signal received from the X-ray detector 107, and stores the generated X-ray image data in the memory 111. The processing circuitry 112 transmits the generated X-ray image data to the image storage apparatus 20 or the medical image processing apparatus 30.

In step S33, the image acquisition function 34a of the medical image processing apparatus 30 acquires the X-ray image data from the X-ray diagnostic apparatus 10 or the image storage apparatus 20 and stores the X-ray image data in the memory 33. The detection function 34b detects the distal end S of the device from the X-ray image data.

In step S34, the operation reception function 34f determines whether an operation for issuing an instruction for the input interface 31 to display the distal end S of the device in the entire image is performed. If an operation for issuing an instruction to display the distal end S is performed (YES in step S34), the processing proceeds to step S35. If an operation for issuing an instruction to display the distal end S is not performed (NO in step S34), the processing proceeds to step S36.

In step S35, the processing circuitry 34 distinguishes the detected distal end S of the device from the X-ray image data stored in the memory 33. The processing circuitry 34 applies a circular shape to the position of the distal end S of the device, as illustrated in FIG. 4B, for example.

In step S36, the detection function 34b of the medical image processing apparatus 30 detects the device line DL from the X-ray image data.

In step S37, the operation reception function 34f determines whether an operation for issuing an instruction for the input interface 31 to display the device line in the entire image is performed. If an operation for issuing an instruction to display the device line is performed (YES in step $37), the processing proceeds to step $38. If an operation for issuing an instruction to display the device line is not performed (NO in step S37), the processing proceeds to step S39.

In step S38, the processing circuitry 34 distinguishes the detected device line DL from the X-ray image data stored in the memory 33. The processing circuitry 34 applies a curve to the device line DL, for example, as illustrated in FIG. 4C.

In step S39, the base point determination function 34c of the medical image processing apparatus 30 determines the base point B of the device from the X-ray image data.

In step S40, the operation reception function 34f determines whether the operation for issuing an instruction for the input interface 31 to display the base point B of the device in the entire image is performed. If an operation for issuing an instruction to display the base point B is performed (YES in step S40), the processing proceeds to step S41. If an operation for issuing an instruction to display the base point B is not performed (NO in step S40), the processing proceeds to step S42.

In step S41, the processing circuitry 34 distinguishes the identified base point B of the device from the X-ray image data stored in the memory 33. The processing circuitry 34 applies a circular shape to the position of the base point B of the device as illustrated in FIG. 4D, for example.

In step S42, the region setting function 34e sets the display region R1, which is a region to be displayed on the display 32, of the X-ray image data.

In step S43, the processing circuitry 34 determines whether an operation for issuing an instruction for the input interface 31 to display the display region R1 in the entire image is performed. If an operation for issuing an instruction to display the region is performed (YES in step S43), the processing proceeds to step S44. If an operation for issuing an instruction to display the region is not performed (NO in step S43), the processing proceeds to step S45.

In step S44, the processing circuitry 34 distinguishes the set display region R1 from the X-ray image data stored in the memory 33. The processing circuitry 34 applies a thick line around the periphery of the display region R1, for example, as illustrated in FIG. 4E.

In step S45, the display control function 34d of the medical image processing apparatus 30 displays, on the display 32, an image of the set display region R1 of the X-ray image data.

In step S46, the display control function 34d displays, on the display 32, the X-ray image data. In the X-ray image data, any one of the distal end S of the device, the device line DL, the base point B of the device, and the display region R1 may be distinguished, or none of these may be distinguished. The display control function 34d may superimpose an image in which at least one of the distal end S of the device, the device line DL, the base point B, and a frame of the display region R1 is identifiable on the X-ray image data, and display the image on the display 32.

While the processing in steps S33 to S46 is described as being performed by the medical image processing apparatus 30, the processing may be performed by the X-ray diagnostic apparatus 10. In other words, the X-ray diagnostic apparatus 10 may perform the entire processing in steps S31 to S46.

According to the second exemplary embodiment, it is possible to support the operation of the device by the practitioner in accordance with a status of a procedure, such as “distance from the distal end” and “enlargement of the displayed region”.

### Third Exemplary Embodiment

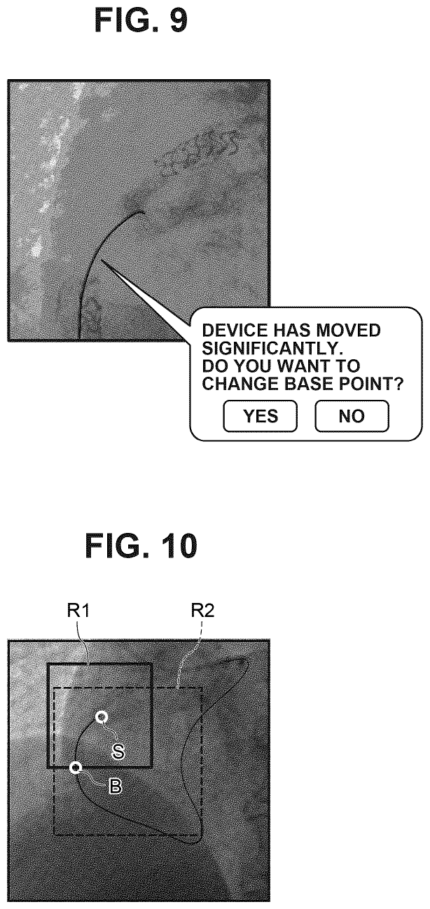

FIG. 9 is a diagram illustrating an example of the display region R1 according to a third exemplary embodiment. As illustrated in FIG. 9, if the amount of change of the device is larger than a predetermined value, the display control function 34d of the medical image processing apparatus 30 may display a pop-up screen with a message “Device has moved significantly. Do you want to change the base point? (Yes) (No)” near the display region R1 displayed on the display 32. In other words, if the amount of change of the device in the set display region R1 is greater than a threshold value, the display control function 34d displays, on the display 32, a screen for proposing a change of the base point B.

Further, if the practitioner clicks (Yes), the display control function 34d may display a mouse pointer so as to move only on the device line to make it easier for the practitioner to change the base point on the device line. The mouse pointer is an example of a pointed position of a pointing device. More specifically, when the operation reception function 34f receives an operation by the practitioner to change the position of the base point B, the display control function 34d displays, on the display 32, a position according to the operation along the device line.

FIG. 10 is a diagram illustrating an example of the X-ray image data according to the third exemplary embodiment. As illustrated in FIG. 10, if it is desired to enlarge a display range regardless of the position of the base point B, the region setting function 34e may set an enlarged region R2 separately from the display region R1 in which the base point of the device is located. For example, when instructed by the practitioner to enlarge the display range of the display region R1, the region setting function 34e sets the enlarged region R2 including at least a portion of the display region R1. The display control function 34d displays, on the display 32, an image of the enlarged region R2 of the X-ray image data together with the image of the display region R1. In such a case, the practitioner can easily check the image of a wide range including at least the portion of the display region R1, and thus, it is possible to improve the efficiency of the procedure.

### Fourth Exemplary Embodiment

In detecting the distal end S of the device or the device line DL, a plurality of devices may be detected. In such a case, the display control function 34d of the medical image processing apparatus 30 may collectively display, on the display 32, images of display regions R1 of the plurality of devices. Alternatively, the display control function 34d of the medical image processing apparatus 30 may display, on the display 32, a screen for selecting a device to be the target of the display region R1.

By combining with electrocardiogram-gated irradiation (X-ray imaging in the same phase as the heartbeat, phase synchronization), the image acquisition function 34a of the medical image processing apparatus 30 acquires an X-ray image in which the movement due to the heartbeat is substantially stopped. Therefore, there is almost no movement due to heartbeat, and only a movement due to breathing is to be suppressed, and thus, a range of movement of the device is limited, and it is possible to increase the accuracy of stopping an unnecessary movement by the fourth exemplary embodiment. Such a feature may be combined with respiratory-gated irradiation.

According to at least one of the exemplary embodiments described above, it is possible to appropriately display a device without a marker pair in the X-ray image.

The image acquisition function 34a and the image acquisition function 112a are examples of an acquisition unit. The detection function 34b and the detection function 112b are examples of a detection unit. The base point determination function 34c and the base point determination function 112c are examples of a base point determination unit. The display control function 34d and the display control function 112d are examples of a display control unit. The region setting function 34e and the region setting function 112e are examples of a setting unit. The operation reception function 34f and the operation reception function 112f are examples of a reception unit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured:
to sequentially acquire an X-ray image including a device inserted into a subject;
to detect a distal end of the device on the X-ray image;
to determine a position on the device distant from the distal end, as a base point; and
to generate an image in which at least a position of the base point is located at a same position or within a predetermined range including the position, among a plurality of X-ray images, and display the image on a display.

2. The medical image processing apparatus according to claim 1, wherein the processing circuitry is configured:
to set a display region for displaying at least a portion of the device from the distal end to the base point,
to generates an image of the display region and displays the generated image on the display so that, for each of the plurality of X-ray images acquired sequentially, the base point is located at a same position or within a predetermined range including the position within the display region.

3. The medical image processing apparatus according to claim 2, wherein the processing circuitry is configured:
to display, on the display, the image of the display region superimposed on a predetermined position of the X-ray image.

4. The medical image processing apparatus according to claim 2, wherein the processing circuitry is configured:
to detect a device line being a linear shape of the device, from the X-ray image; and
to determine, as the base point, a position located at a predetermined distance from the distal end along the device line.

5. The medical image processing apparatus according to claim 4, wherein the predetermined distance is determined in advance according to at least one of an X-ray imaging condition and a protocol.

6. The medical image processing apparatus according to claim 5, wherein the processing circuitry is configured:
to convert the predetermined distance determined in advance into a number of pixels on the X-ray image; and
to determine the base point based on the distal end and the number of pixels.

7. The medical image processing apparatus according to claim 6, wherein the processing circuitry is configured:
to convert the predetermined distance into the number of pixels on the X-ray image based on three-dimensional information of the device.

8. The medical image processing apparatus according to claim 4, wherein the processing circuitry is configured:
to change the predetermined distance according to an amount of change of the device in the display region.

9. The medical image processing apparatus according to claim 8, wherein the processing circuitry is configured:
to display, on the display, a screen for proposing a change of the base point when the amount of change of the device in the display region is greater than a threshold value.

10. The medical image processing apparatus according to claim 9, wherein the processing circuitry is configured:
to receive an operation by a user to change the position of the base point,
to display a position according to the operation along the device line on the display.

11. The medical image processing apparatus according to claim 2, wherein the processing circuitry is configured:
to display, on the display, an image in which at least one of the distal end of the device, a device line, the base point, and a frame of the display region is distinguishable superimposed on the X-ray image.

12. The medical image processing apparatus according to claim 4, wherein the processing circuitry is configured:
to determine, as a second base point, a position at a distance different from the predetermined distance from the distal end along the device line; and
to set the display region for each of the plurality of X-ray images sequentially acquired, based on positions of the base point and the second base point.

13. The medical image processing apparatus according to claim 12, wherein the processing circuitry is configured:
to cause the position of the second base point to be at approximately a same position in the display region while changing the position of the base point in accordance with an amount of change of the distal end to generate the image of the display region.

14. The medical image processing apparatus according to claim 2, wherein the processing circuitry is configured:
to set an enlarged region including at least a portion of the display region when the setting is instructed by a user to enlarge a display range of the display region; and
to display, on the display, an image of the enlarged region of the X-ray image together with the image of the display region.

15. An X-ray diagnostic apparatus comprising:
an X-ray tube configured to generate X-rays;
an X-ray detector configured to detect X-rays emitted from the X-ray tube and output a detection signal corresponding to an amount of the detected X-rays;
processing circuitry configured;
to sequentially acquire an X-ray image based on the detection signal, the X-ray image including a device inserted into a subject; and
to detect a distal end of the device on the X-ray image;
to determine a position on the device distant from the distal end, as a base point; and
to align a plurality of the X-ray images, based on positions of the distal end and the base point, and display the plurality of X-ray images on a display.

16. A non-transitory computer-readable storage medium storing a medical image processing program, the storage medium causes a computer to execute processes of;
sequentially acquiring an X-ray image including a device inserted into a subject;
detecting a distal end of the device on the X-ray image;
determining a position on the device distant from the distal end, as a base point; and
aligning a plurality of the X-ray images, based on positions of the distal end and the base point; and
displaying the plurality of X-ray images on a display.
